(19) United States
(12) Patent Application Publication
(10) Pub. No.: US 2003/0045868 A1
(43) Pub. Date: Mar. 6, 2003

(54) Apparatus for conveying a light source to an intravenous needle to kill blood pathogens

(76) Inventor: Joseph DiStefano, St. Petersburg, FL (US)

Correspondence Address: LARSON AND LARSON, 11199 69TH STREET NORTH, LARGO, FL, 33773

(21) Appl. No.: 09/940,996

(22) Filed: Aug. 28, 2001

(51) Int. Cl. A61B 18/18; A61F 11/00; A61B 17/32

(52) U.S. Cl. 606/15; 606/7; 606/167; 606/108

(57) ABSTRACT

A tip of a needle is inserted into large vein of a patient. The needle is mounted in a butterfly device axially engaged to a generally cylindrical housing containing a Y-connector. Saline solution is fed to the needle through the Y-connector to the housing. A quartz optic fiber is fed through the housing and needle to the tip of the needle in the patient's venous system. A source of UV and visible light directs both UV and visible light alternatively through the optic fiber to the patient's venous system to kill pathogens in the venous system.

### FIELD OF THE INVENTION

This invention relates to an apparatus emitting light rays to kill pathogenic organisms. More particularly, it refers to an apparatus for the conveying of alternating UV and visible light through a quartz fiber optic cable to a needle inserted into a patient's venous blood.

### BACKGROUND OF THE INVENTION

It is well known to use UV light to kill pathogens in water. Many systems exist to expose water to UV light with the object of destroying pathogens in the water. It also is well known to guide fiber optic instruments into arterial blood vessels. See U.S. Pat. No. 4,830,460 where the ultraviolet laser energy is used for the ablation of atherosclerotic plaque; U.S. Pat. No. 5,053,033 where an optical fiber delivers UV radiation to a blood vessel site following angioplasty to kill aortic smooth muscle cells at the site; U.S. Pat. No. 5,891,082 where a catheter body has a light-emitting section at a distal end for curing a stent delivered to a blood vessel site; U.S. Pat. No. 6,117,128 where laser energy coupled to an optical fiber and transported by a catheter to treat vascular thrombosis disorders in the brain; and U.S. Pat. No. 6,187,030 where a flexible fiber optic bundle connected to a light source is used for treatments of internal and external diseases.

In spite of the wide use of UV light by the medical profession no apparatus has been developed for cleansing blood of pathogens such as fungi, virus, bacteria and molds. Such an apparatus is in critical need.

### SUMMARY OF THE INVENTION

This invention is the discovery of an apparatus for destroying pathogens in the venous blood of a patient. A rear portion of a needle is mounted in a butterfly housing with a tip extending outwardly. The tip of the needle in the apparatus is inserted into a large vein of a patient. A saline stream flows through the needle arm of a Y-housing and through the needle to its tip. A first portion of a quartz optic fiber passes through another arm of the Y-housing and through the needle to a tip. A second end of the quartz optic fiber is connected to a first end of a fiber optic bundle which is connected at a second end to a source of both UV and visible light. The apparatus is used to expose venous blood to about thirty minutes of UV light wavelength of 200-450 nanometers and then for about thirty minutes to visible light.

### BRIEF DESCRIPTION OF THE DRAWINGS

The invention can be best understood by those having ordinary skill in the art by reference to the following detailed description when considered in conjunction with the accompanying drawings in which:

FIG. 1 is perspective view of a needle, an optic fiber and a saline water source housed in a butterfly throw away device.

FIG. 2 is a perspective view of the device of FIG. 1 prior to inserting the needle in a vein of a patient.

FIG. 3 is a perspective view of the device taped to a patient's arm with the needle inserted into the patient's vein.

FIG. 4 is a schematic view of the apparatus of this invention.

FIG. 5 is an open conduit view of the saline drip from the saline solution source.

### DETAILED DESCRIPTION OF THE INVENTION

Throughout the following detailed description the same reference numerals refer to the same elements in all figures.

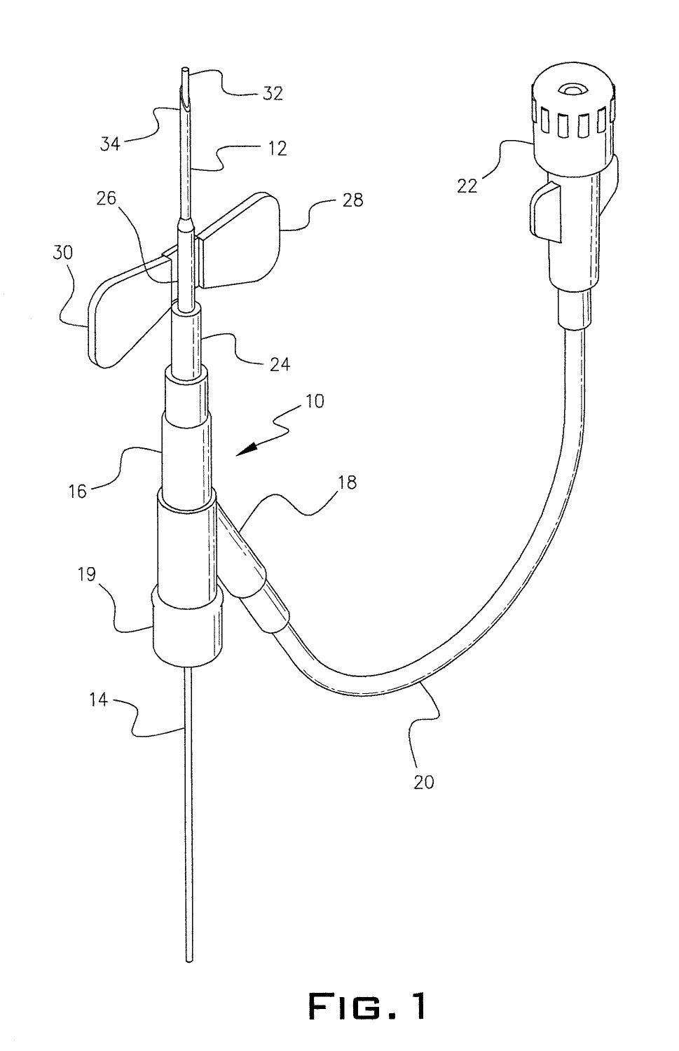

Referring first to FIG. 1, the housing 10 is a plastic disposable device in a Y-configuration having a first arm 18 and a second arm 19 leading to a cylindrical body 16. A quartz optic fiber 14 is threaded through the arm 19 of the housing 10. Arm 18 is connected to a saline solution delivery conduit 20 from a connector 22. The cylindrical body 10 has a cylindrical end portion 24 receiving a hub 26 integral with butterfly wings 28 and 30. The hub 26 retains an upper portion of needle 12 in place. The optic fiber 14 is threaded from arm 19, through body 16 and through the needle 12 positioned in hub 26. An end 32 of the optic fiber 14 is visible at the tip 34 of needle 12.

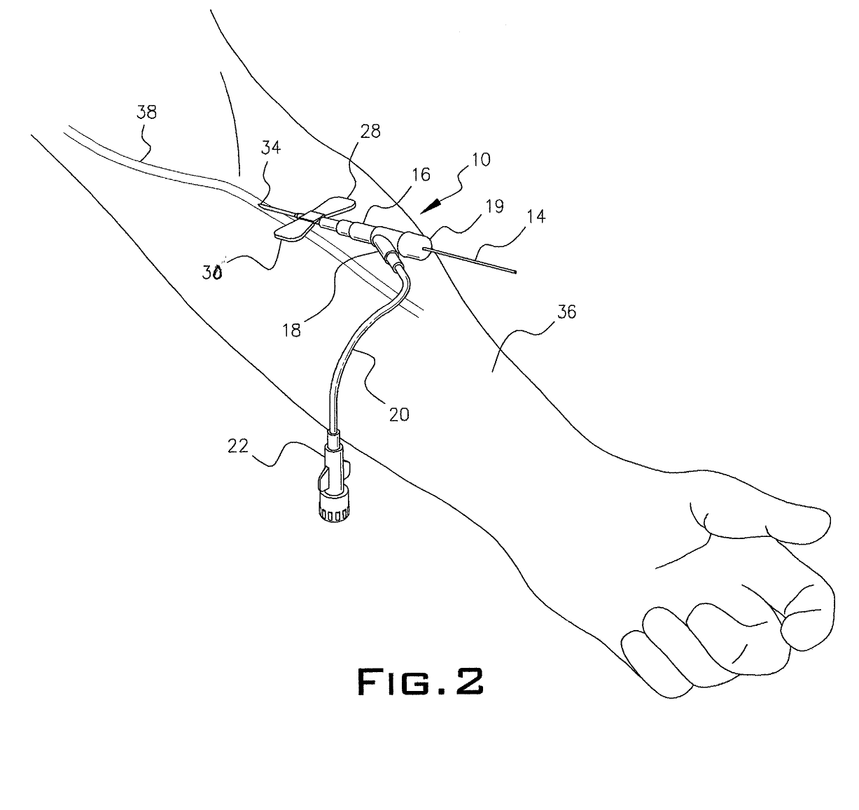

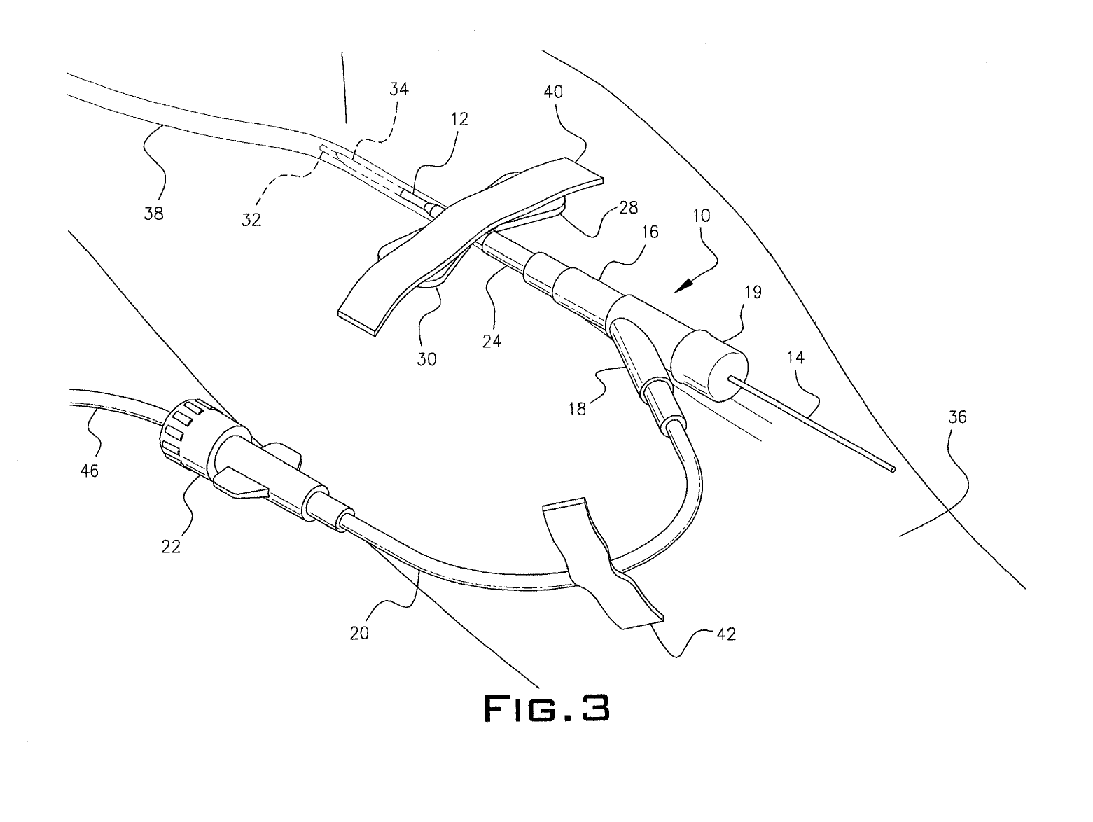

In FIG. 2 the arm 36 of a patient is shown with a vein 38 about to be penetrated by the point 34 of needle 12. After the needle 12 is inserted into vein 38 as shown in FIG. 3 the butterfly wings 28 and 30 are taped 40 to arm 36. In like manner the saline water conduit 20 is taped 42 to the patients arm 36.

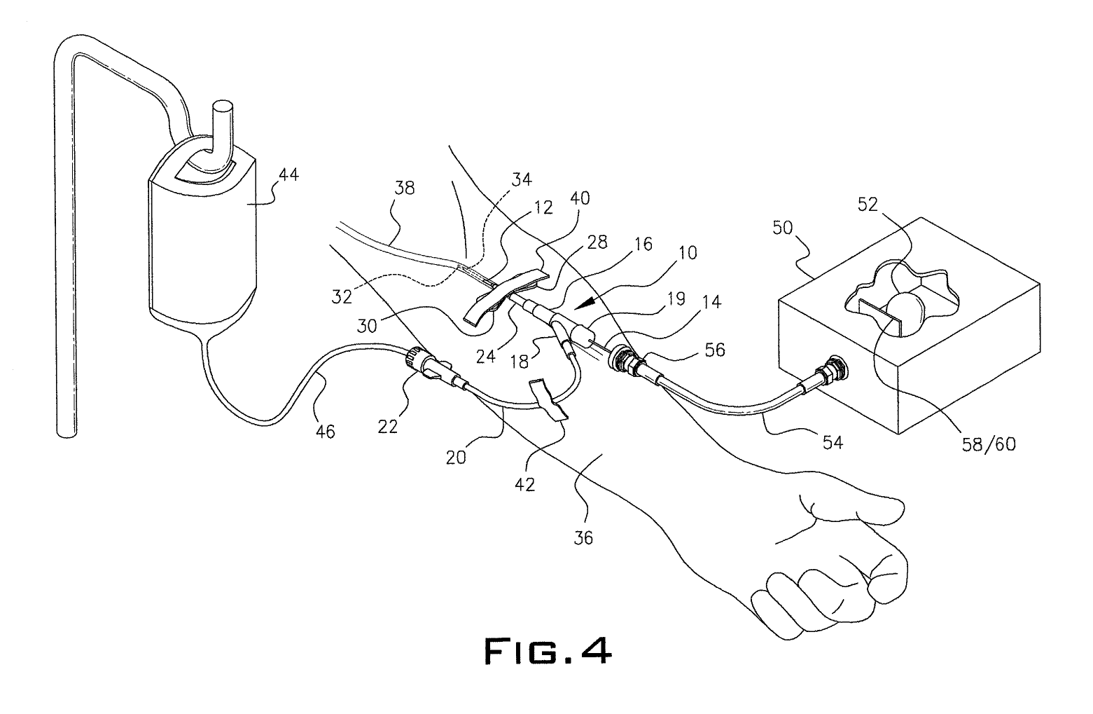

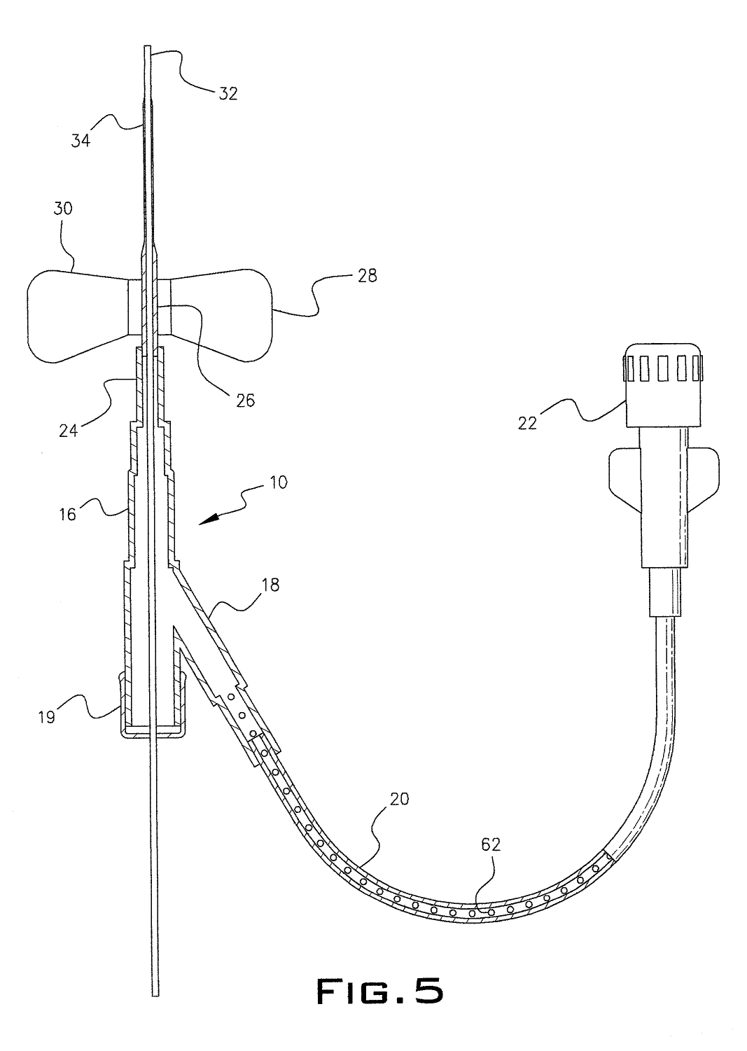

As seen in FIGS. 4 and 5, a saline solution bag 44 has a conduit 46 leading to a connector 22 which determines the drip rate 62 to needle 12. A continuous rate of one drop per second is sufficient to keep tip 34 and end 32 of the optic fiber 14 clear of blocking blood cells. The UV or visible light source eminates from a box 50 containing a bulb 52 which can have rotating filter, 58 and 60 around the bulb 52 to permit either UV or visible light to travel through a bundle of quartz optic fibers 54 to a connector 56 engaging to optic fiber 14.

The needle 12 is usually about 19 gauge for an adult and about 25 for a pediatric patient. The diameter of the quartz optic fiber 14 will be determined by the size of the needle.

The treatment usually lasts one hour with the first thirty minutes under UV light of 200-450 nanometer wavelength and then for thirty minutes at visible light of 450 to 1100 nanometer wavelength.

The butterfly wings 28 and 30 together with its hub is made from a typical medically acceptable plastic. The housing 10 and the conduits 20 and 46 also are made from a medically acceptable plastic.

Pathogens in the venous blood supply killed by the use of the apparatus include bacteria, virus, fungi, molds and other unclassified pathogens.

The above description has described the specific apparatus of this invention. However, it will be within the skill of one having ordinary skill in the medical field to make modifications without departing from the spirit and scope of the underlying inventive concept of this apparatus.

Having described the invention in detail the following is claimed:

1. An apparatus for treating venous blood of a patient to control the presence of pathogens in the blood stream compromising:
(a) a Y-shaped housing having an annular internal diameter,
(b) a terminal end of the Y-shaped housing enclosing the hub of a needle housing,
(c) a needle having a first end mounted in the hub and a second end tip protruding outside the hub for inserting into a patient's vein in which a blood stream is flowing,
(d) a saline solution bag with a conduit from the bag directed to a first arm of the Y-shaped housing,
(d) a first portion of a quartz optic fiber passing through a second arm of the Y-shaped housing and through the needle to the tip,
(f) a second portion of the quartz optic fiber connected by a quartz fiber optic bundle to a light housing containing a source of UV and visible light whereby the UV and visible light is conducted to the tip of the needle.

2. The apparatus according to claim 1 wherein the terminal end of the Y-shaped housing has an exterior annular diameter decreasing in width from the first and second arms.

3. The apparatus according to claim 1 wherein the needle housing has a butterfly wing configuration attached to an exterior surface of the hub.

4. The apparatus according to claim 1 wherein the needle is about 19 gauge.

5. The apparatus according to claim 1 wherein the conduit from the saline solution bag passes through a connector monitoring the flow of solution.

6. The apparatus according to claim 5 wherein the connector is adapted to permit a flow of about one drop per second.

7. An apparatus for destroying pathogens in a venous blood vessel comprising:
(a) a housing having a first and second arm leading to a cylindrical body, an end portion of the body distal from the first and second arms enclosing an annular needle housing,
(b) a needle having a first portion mounted in the annular needle housing and a second portion extending outwardly to a tip for inserting into a vein of a patient,
(c) a saline solution bag having a conduit leading to the first arm,
(d) a first end portion of a quartz optic fiber passing through the second arm and through the needle to the tip, and
(e) a second portion of the quartz optic fiber connected through a connector to a first end of a fiber optic bundle, the fiber optic bundle connected at a second end to a source of UV and visible light whereby the UV and visible light is conducted to the tip of the needle, while saline solution is flowing through the needle to the tip.

8. An apparatus according to claim 7 wherein the annular needle housing has a butterfly wing configuration attached to an exterior surface of the housing.

9. An apparatus according to claim 7 wherein the needle is about a 19 gauge.

10. An apparatus according to claim 7 wherein the conduit from the saline solution bag passes through a connector monitoring the flow of solution.

11. The apparatus according to claim 10 wherein the connector is adapted to permit a flow of about one drop per second.

12. The apparatus according to claim 7 wherein the source of UV and visible light is a lighted bulb separated from the second end of the fiber optic bundle by either a UV or visible light filter.
